(19) United States
(12) Patent Application Publication
(10) Pub. No.: US 2003/0103863 A1
(43) Pub. Date: Jun. 5, 2003

(54) High pressure sterilising of sensitive active principles

(76) Inventors: Luc Grislain, Blanqeufort (FR); Bruno Vallayer, Broue (FR); Gerard Demazeau, Gradignan (FR); Alain Largeteau, Cestas (FR); Gilles Lemagnen, Tresses (FR); Yohan Rigaldie, Bordeaux (FR)

Correspondence Address: YOUNG & THOMPSON, 745 SOUTH 23RD STREET 2ND FLOOR, ARLINGTON, VA, 22202

(21) Appl. No.: 10/182,372

(22) PCT Filed: Jan. 29, 2001
(86) PCT No.: PCT/FR01/00270

(30) Foreign Application Priority Data
Jan. 27, 2000 (FR) 00/1059

(51) Int. Cl. A61L 2/00

(52) U.S. Cl. 422/1; 422/33; 422/39

(57) ABSTRACT

The invention concerns a method for sterilising at least a sensitive active principle, characterised in that said at least active principle is treated at pressure levels ranging between 3000 and 6000 bars and at temperatures ranging between −30° C. and +25° C. The method consists in treating directly the active principle in the pulverulent medium wherein it is contained.

The present invention relates to a process for sterilization by high pressure of sensitive active principles, particularly peptides, oligonucleotides or proteins.

There is known the sterilization of agro-alimentary products by pressure, such as the process described in European patent application 89 4440. This process permits treating liquids continuously by treatment of said liquids in a series of stages in which the pressures increase progressively. When the liquid reaches the last stage at very high pressure, the liquid is subjected to rapid depressurization, which ruptures the microorganisms.

Other processes provide for combining very high pressure with high temperatures. Thus the weakly acid foodstuffs, already packaged, are subjected to a prepressurization temperature; then, during a very brief time, the whole is brought to a very high pressure, which causes the temperature instantaneously to rise to high values. The pressure is returned to the starting pressure at the same time that the temperature immediately falls.

These processes permit destroying microorganisms such as virus, bacteria and mold in liquids, because the liquids perfectly transmit the pressure exerted, in an isostatic way.

German patent application DE-A-19 905159 discloses a process for the sterilization of substances or active principles that are to be introduced into medications, at temperatures comprised between 20 and 9000 bars and at temperatures comprised between 25° C. and 200° C.

Such temperatures result in degradation of certain active principles and the object is generally the sterilization of very resistant active principles.

International patent application WO 98 47503 discloses a process to sterilize preparations based on ibuprofen. The object is to carry out high pressurization at 3,000 to 10,000 bars, whilst remaining at temperatures lower than the melting temperature of ibuprofen.

In French patent application FR-A-2 740 993, there is mentioned an isostatic press to treat loaded liquids so as to increase the time of preservation, particularly in the agro-alimentary field.

By contrast, from an economic standpoint, such processes are poorly adapted for low added value goods, because they give rise to important investment which is difficult to amortize.

There are known other means for sterilization by radioactive processes, but in this case, if certain radiation can be satisfactory, it gives rise to complicated problems as to their handling and above all raises questions as to the risks to the environment, connected to their use.

Sterilizing membranes are also a good means to retain undesired particles, but it is also necessary that the separation be suitable and that it is possible to separate mechanically the different categories. Generally speaking, industrial regulations required for filtration, render them less practical to use and limit them to liquids alone.

The most usual technique consists in sterilizing by heat under pressure as indicated above, or simply with heat at ambient pressure. This method is applicable only to products which are resistant to temperature, however numerous active principles are sensitive to heat. The active principles arise particularly from biotechnology, such as peptides or molecules from research on DNA.

The process according to the present invention provides steps permitting sterilizing, by a high pressure treatment, sensitive active principles, without degrading them, and under conditions of operation suitable for industry. Such a process, applied to therapeutic entities of high added value, is economically suitable but there remains a prejudice which holds that sensitive active principles are degraded by subjection to very high pressures and that the use of very high pressures as a bactericidal and viricidal means, is not suitable.

Contrary to what is known to the prior art, temperature, in negative ranges, permits reinforcing the effects of sterilization under pressure.

It will also be noted that the application of this sterilization process to solids in divided form is particularly illustrative of the capability of sterilizing products other than liquids.

To this end, the sterilization process of at least one sensitive active principle, according to the invention, is characterized in that the at least one active principle is treated at pressures comprised between 3,000 and 6,000 bars and at temperatures comprised between −30° C. and +25° C.

More particularly, the active principle that is treated is of a molecular weight less than or equal to 170,000 g/mol.

There are retained specifically the peptides, insulin and antibodies.

Preferably, the process is conducted at negative temperatures down to −30° C. and even directly on a formulation containing the active principle in pulverulent medium.

So as to achieve the desired sterilizing effect, the pressure and temperature are adjusted to achieve a logarithmic reduction of contamination of the microorganisms greater than 6.

The process will now be described by giving several examples and results of tests, in connection with the accompanying drawings, in which the different figures represent:

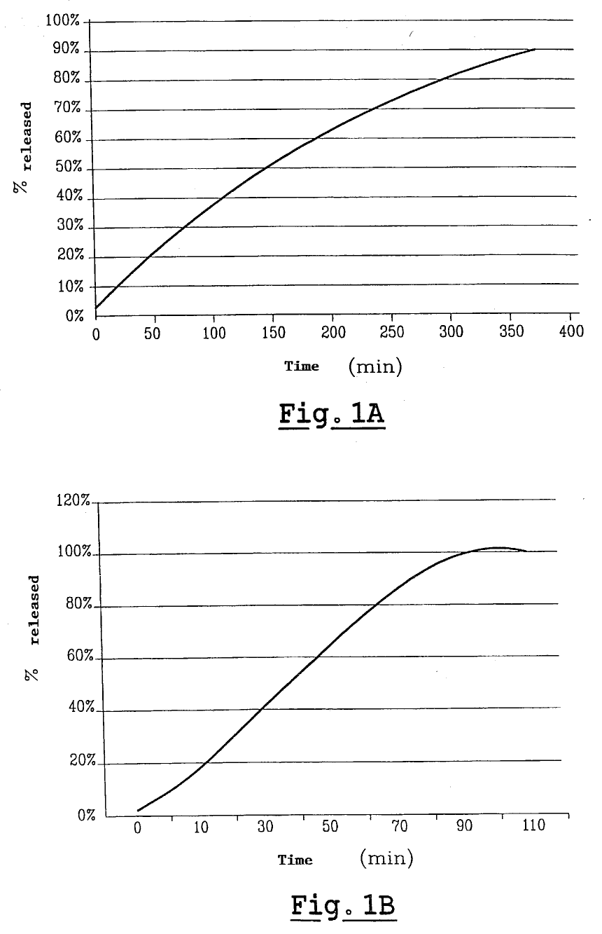

FIG. 1A: kinetic curve of dissolution of ibuprofen in tablet form, obtained by isostatic pressing at high pressure,

FIG. 1B, kinetic curve of comparative dissolution of ibuprofen as a tablet, obtained by uni-axial pressing under high pressure,

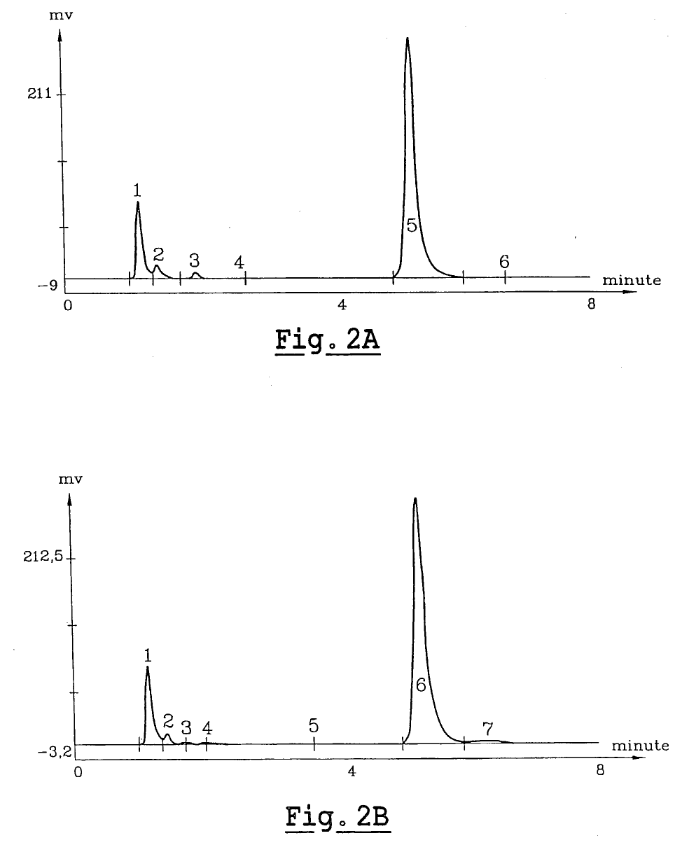

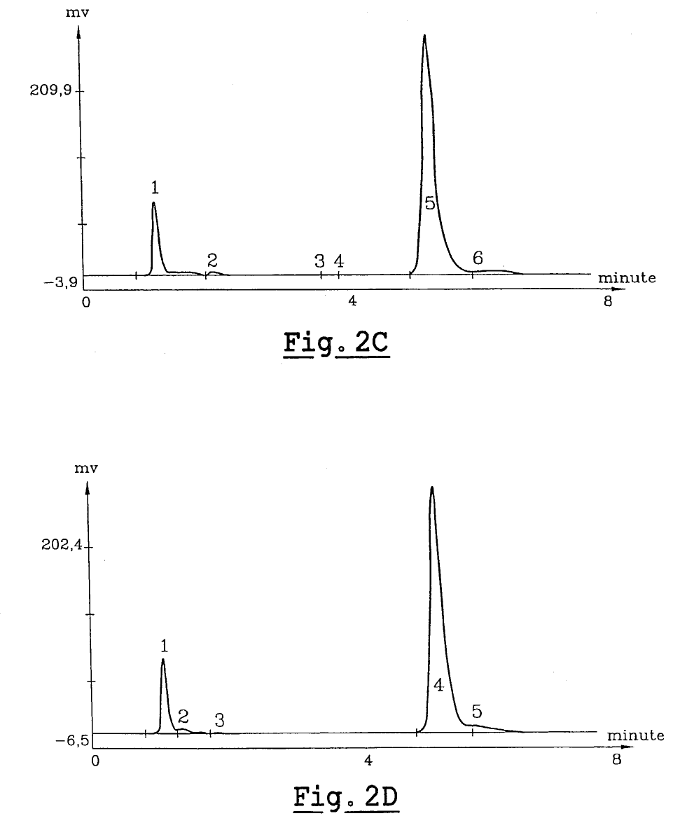

FIGS. 2A-2D: chromatographic curves of insulin after high pressure treatment,

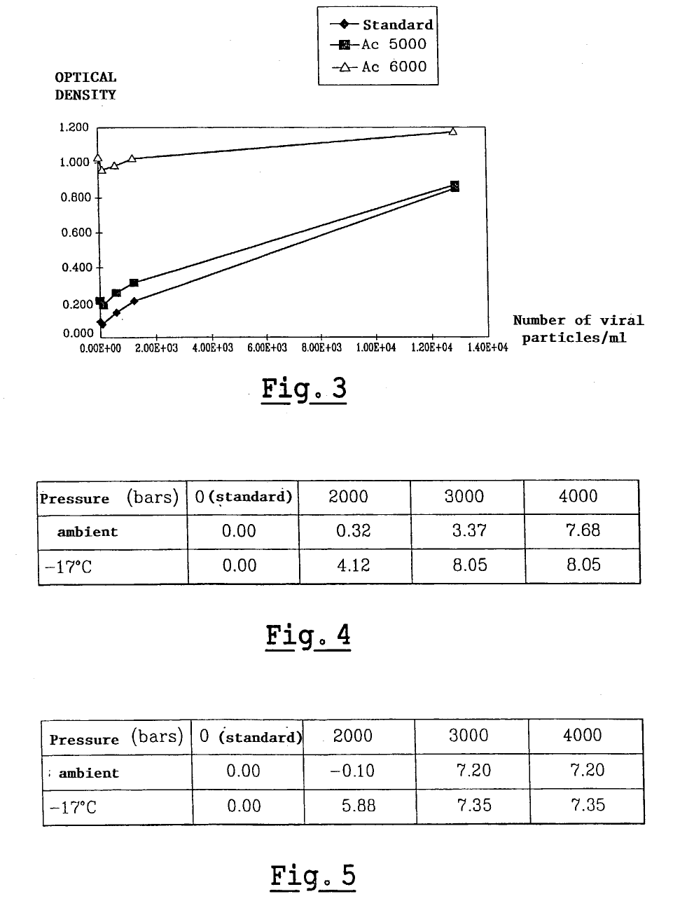

FIG. 3: curves of immunological activity of the antibodies treated under high pressure,

FIG. 4: table of results obtained with tests on Pseudomonas aeruginosa

FIG. 5: table of results obtained with tests on Candida albicans

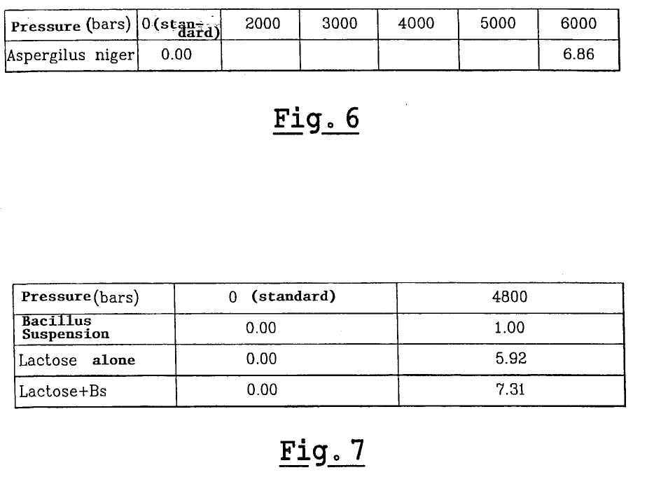

FIG. 6: table of results obtained with tests on sporulated Aspergillus niger, and

FIG. 7: table of results obtained with tests on Bacillus subtilis, directly on a pulverulent medium.

There will be first of all defined the possibilities of treatment of fragile molecules by high pressure, by showing that the structure of the active principles remains unchanged after subjection to such pressures.

There is meant by fragile elements, molecules such as proteins, antibodies, peptides, enzymes, but also medical vectors such as microspheres, nanocapsules or liposomes. These fragile elements cannot resist high temperature or radiation.

It is known that during isostatic pressing, very little energy is transmitted to the treated products, which preserves their potential.

The description which follows is applicable to the sterilization of powders, such as powders for extemporaneous preparations destined to be injected. Moreover, it is possible to produce directly by pressing, tablets from powders, in the desired form, and sterilized to be implantable. It is necessary only to provide a matrix suitable to obtain a form and a desired mass, in a reproducible manner.

Tests that have been carried out were with a non-steroidal anti-inflammatory of the group of propionics, derived from carboxylic acid, ibuprofen, in the form of a base.

More particularly, the following formulation was used:

IBUPROFEN: 60% (produced by the Knoff company under UPSA product code)

LACTOSE: 35% (produced by the Seppic company under product code A16 M07)

AEROSIL 200: 0.5% (produced by the Degussa company under the product code M01 03 E330)

MAGNESIUM STEARATE 200: 0.5% (produced by the Cpf company under the product code M01 03 E677)

TALC: 4.0% (produced by the Cpf company under the product code M01 03 E044)

Tablets were formed by isostatic pressing at 2000 bars.

There were carried out tests of dissolution with an agitation of 100 rpm, at 37° C. and a pH of 7.2.

The curve of FIG. 1A shows the dissolution kinetic in the case of isostatic pressing, which is practically linear, but with a prolonged duration of release of the active principle of nearly 4 hours.

By way of comparison, there is carried out a uni-axial compression with the same formulation and with the same exerted pressure, the corresponding curve being indicated in FIG. 1B. It will be noted that all of the ibuprofen is released in less than two hours.

A first advantage of isostatic pressing is an improved dissolution kinetic.

Complementary tests were conducted to show the harmlessness of the treatment by high pressure on the fragile elements. In this case, these elements were placed in solution or in the form of powder.

There is thus used for this purpose fragile elements of molecular weight less than or equal to 170,000 g/mol such as peptides, proteins and antibodies and more particularly:

a decapeptide: gramicidin (PM=1 140 g/mol), in the form of powder.

insulin 40 UI (PM=5,800 g/mol), and

a monoclonal antibody such as the grippe anti-virus antibody A (PM=150,000 g/mol).

a) The peptide, in this case Gramicidin, is subjected to a high pressure treatment at 5,000 bars, and its purity was also tested by HPLC, the variations remaining less than 1%. The high pressures have no influence on the structure.

b) Insulin was selected at 40 UI, commercial form, and diluted to 1/100 at pH 2.5. For the measurement, passage is carried out with a flow rate of 1 ml/min in an HPLC (High Pressure Liquid Chromatography) apparatus.

Different specimens were subjected, at ambient temperature and at a negative temperature of −20° C., to:

atmospheric pressure for the standard,

4,000 bars for 10 minutes,

5,000 bars for 10 minutes, and

6,000 bars for 10 minutes.

The specimens, after treatment, are then analyzed at 214 nm in a chromatographic apparatus with automatic injection. There are obtained the following results, shown in FIGS. 2A, 2B and 2C and 2D.

It will be seen that the different curves of the specimens subjected to sterilization by high pressure treatment, show the same peaks with substantially the same intensities and centered on the same temperature ranges as in the case of the standard at atmospheric pressure, including when the temperature is negative.

There are no noticeable modifications or alterations of the structure of the product.

c) As to monoclonal antibodies, there were tested monoclonal antibodies directed against the grippe A virus of a molecular weight of 150,000 g/mol, which were subjected to high pressures of 4,000, 5,000 and 6,000 bars for 10 minutes at ambient temperature.

An ELISA test shows that the immunological activity of the antibodies treated up to 5,000 bars is preserved, whilst this activity tends to decrease beyond 6,000 bars. This is shown by the curves of FIG. 3.

Because the high pressures do not degrade the active principles, the following tests were then carried out to show the effectiveness of the treatment as to microorganisms.

As there is no microbiological indicator of normalized effectiveness to qualify sterilizing treatments, there were selected four pathogenic microorganisms to constitute a specimen that would be the most representative, these microorganisms being in liquid medium:

one gram—: Pseudomonas aeruginosa,

a yeast: Candida albicans, and

a mold spore: sporulated Aspergillus niger.

According to European pharmacology, a sterilizing treatment requires a decrease by 6 logs of the population of micro-organisms.

Tests on: Pseudomonas aeruginosa

The results are gathered in the table of FIG. 4, which shows the logarithmic reduction. It will be seen that the pressure always produces a sterilizing effect when reaching a high pressure, 4,000 bars.

It will be noted in these tests that the manipulations were carried out at ambient temperature and under refrigeration, −17° C. Surprisingly, there is seen an increase in the sterilizing effect of pressure and after 3,000 bars, the desired sterilizing effect is reached.

Tests on Candida albicans

This microorganism is particularly sensitive to pressure, but it is again seen that the effect of low temperatures which reinforce the action and permit achieving beyond 2,000 bars, practically the 6 log differential. Table of FIG. 5.

Tests on sporulated Aspergillus niger

The tests show that it is necessary to reach 6,000 bars to achieve a logarithmic amount of reduction greater than 6, assisted by the effects arising from low temperature at −17° C. Table of FIG. 6.

Other tests have been conducted directly on pulverulent media.

The pulverulent medium used is lactose, an excipient which is found in the production of numerous pharmaceutical pills or in powders for the preparation of injectibles.

The implanted strain is Bacillus subtilis.

So as to determine the background noise of lactose which could disturb measurement, there is carried out a powder specimen test. This permits taking account of the possible contamination arising from lactose.

Each specimen is placed in a sealed envelope and subjected to high pressures, 4,000 bars in this instance, by isostatic pressing.

The two other specimens comprise, for the first, lactose with Bacillus subtilis bacteria, in lyophilized form, and for the second, a same dose of Bacillus subtilis bacteria, lyophized but simply in a diluant so as to expose only the bacterial material to the sterilizing action.

The results are shown in the table of FIG. 7. They show that the lactose already contain microorganisms and that these residents were destroyed like the added load, the logarithmic reduction being 7.31.

There is noted less effectiveness of treatment by high pressures on the microbial load in liquid medium.

It will also be noted that the treatments under pressure with a negative temperature permit improving the performance of sterilization in particular when the temperature is brought down to −17° C., even −20° C. or −30° C., instead of ambient temperature, during treatment under pressure.

Also, the sterilization treatment under pressure, between 3,000 and 6,000 bars, particularly of powders, according to the present invention, can be conducted over a temperature range covering −30° C. to +25° C. This treatment does not affect the retained active principles.

1. Process of sterilization of at least one sensitive active principle, characterized in that the at least one active principle is subjected to an isostatic pressure at pressures comprised between 3,000 and 6,000 bars and at temperatures comprised between −30° C. and +25° C.

2. Sterilization process according to claim 1, characterized in that the active principle treated has a molecular weight less than or equal to 170,000 g/mol.

3. Sterilization process according to claim 2, characterized in that the active principle is of the peptide family.

4. Sterilization process according to claim 2, characterized in that the active principle is insulin.

5. Sterilization process according to claim 2, characterized in that the active principle is an antibody.

6. Sterilization process according to any one of the preceding claims, characterized in that it is preferably conducted at negative temperatures down to −30° C.

7. Sterilization process according to any one of the preceding claims, characterized in that the isostatic pressure is applied directly to a formulation containing the active principle in pulverulent medium. 8. Sterilization process according to any one of the preceding claims, characterized in that the pressure and temperature are adjusted to achieve a logarithmic reduction of contamination by microorganisms greater than 6.
